(19) United States
(12) Patent Application Publication
(10) Pub. No.: US 2007/0212718 A1
(43) Pub. Date: Sep. 13, 2007

(54) APPARATUS AND METHODS FOR APPLICATIONS OF GENOMIC MICROARRAYS IN SCREENING, SURVEILLANCE AND DIAGNOSTICS

(75) Inventor: Clark TIBBETTS, Sperryville, VA (US)

Correspondence Address: OBLON, SPIVAK, MCCLELLAND, MAIER & NEUSTADT, P.C., 1940 DUKE STREET, ALEXANDRIA, VA, 22314, US

(73) Assignee: Tessarae Inc., Potomac Falls, VA (US)

(21) Appl. No.: 11/684,371

(22) Filed: Mar. 9, 2007

Related U.S. Application Data
(60) Provisional application No. 60/780,651, filed on Mar. 9, 2006.

(51) Int. Cl. C12Q 1/68; G06F 19/00

(52) U.S. Cl. 435/6; 702/20

(57) ABSTRACT

This disclosure describes combinations of apparatus and methods to comprise a system for broad use and effective application of analytical genomic microarrays for screening and surveillance. The methodology abandons reliance on typical volumes of peripheral blood samples obtained by phlebotomy, in preference for protocols enabling collection, stabilization, archives extraction and purification of small volumes as obtained from a finger prick. Recommended processing protocols from such starting material favor preparation of sufficient quantities of RNA or DNA of sufficient quality for subsequent steps of targeted amplification and fluorescent labeling. A strategy is offered for effective integration of capabilities for genotype and phenotype analysis on the same microarray layout. These gene expression re-sequencing arrays (GXR) are well suited for screening and surveillance applications.

### CROSS REFERENCE TO RELATED CASES

This application claims priority to U.S. 60/780,651, filed on Mar. 9, 2006, which is hereby incorporated by reference in its entirety.

### BACKGROUND OF THE INVENTION

1. Field of the Invention

This invention relates to apparatus and methods combined to comprise a system for broad use and effective application of analytical genomic microarrays for screening and surveillance. Such applications do not exclude potential uses of the invention for clinical diagnostics and may indeed lead to powerful alternative approaches to multiplex diagnostic analysis.

Applications for this invention emphasize capabilities to provide decision quality information on site and in near real time, at low cost and high added value consistent with intentions to deploy the technology for widespread high volume use. Some applications may provide results of immediate and permanent informatic value as single assays or tests, however the technology is particularly suited to parallel multiple assays (as across different sample source sites from a single individual or area and time frame), or to serial multiple assays (as temporal or longitudinally tracked assay results representing samples from the same individual or site collected over specific intervals of time).

For medically related applications, the cost and benefits of performing tests using the technology of this invention must be balanced with respect to patient/participant risk—for example, the system must be operable with patient specimen material obtained by effectively non-invasive methods (fingerprick blood, buccal swab, etc.); the results should offer added medical value to care provider or health or quality of life value to the patient; or the test results should effectively displace accepted best practice methodologies by offering at least equivalent quality of information at lower cost.

2. Discussion of the Background

Although genomic microarray technologies are notionally similar for most applications the requirements and logistical issues in screening and surveillance impose particular constraints that are different from medical diagnostics, prognostics and treatment planning. Screening and surveillance methods for cancer, for example, are premised on generalized risk factors and specific complaints, but do not necessarily identify an a priori target tissue and site for increasingly detailed evaluation.

A primary value for screening and surveillance is to detect, identify and track progression of and risk factors for individuals and to convolve such data with respect to broad statistical knowledge and inference from families and communities. This supports partitioning of limited resources for increasingly specific testing to individuals revealed to have elevated specific risks (probable cause for significant value from performance of the assay or test).

Screening and surveillance capabilities will find applications beyond medical venues including detection and identification assessments in forensics, epidemiology, environment, agriculture, food chain products, industrial materials, waste processing and disposal.

Broad applications of effective technologies, such as provided by this invention, provide baseline capabilities that enable heightened preparedness for anticipated risks and incidental events of concern for public health, homeland security and biological defense.

Research has highlighted applications of microarrays using nucleic acid probes for detection and identification of specific sequences—genotype—and for profiling patterns of (m)RNA as indicators of relative levels of gene expression—phenotype—with a broad swath of potential applications for medicine and other fields.

A key reference (Cummings, C. A. and Relman, D. A. (2000) Emerging Inf Dis 6, 513-525) cites the use of microarrays for the study of host-pathogen interactions (microbiology). Arrays representing DNA (or RNA) sequences of multiple microbial genomes enable detection and identification of which organisms, singly or in mixture, are present in a clinical specimen. The method based on microarray re-sequencing provides unequivocal evidence based on dozens to hundreds of bases of contiguous sequence for the presence of one or more organisms, at levels of discrimination that permit forensic identification of the specific strain or variant of each present organism. Sensitivity can be high if organisms are present at the time of collection at the site sampled to provide the specimen, using nucleic acid amplificiation (such as polymerase chain reaction, PCR) to evidence even few genomic copies of the organism within the limits of sample volume.

Such sensitivity and specificity imply extraordinarily low rates of false positive for detection and identification, and they invite exculpatory inferences from parallel negative results from the same microarray. However it is important to recognize that false negative results are more likely to result from variations in the natural history of the pathogen in relation to the host than from technical failure of these methods. Was the right tissue or fluid sampled at the right time in the temporal course of the cycle of exposure, infection and recovery?

Microarrays offer a complementary view to host-pathogen interactions by evaluation of the gene expression of the host immune cells, in their particular state of response to the insult of the pathogen. It is possible such analysis of host gene expression can reveal not only the stage of infection, but indicate to some extent the nature and identity of the etiologic agent(s)—as in distinguishing infection by viruses or bacteria of different types.

Another foundation reference (Golub, et al (1999) Science 286, 531-537) uses a subset of genes identified from global gene expression profiling to discriminate between pathologically distinct forms of acute leukemia with very different preferred approaches to therapy.

Another foundation reference (Solus, et al (2004) Pharmacogenomics 5:895-931) describes the DNA sequence variations individuals with respect to genotypes among multiple cytochrome P450 genes that are important in the phamacokinetics activation and breakdown of many therapeutic agents. This technology is most recently manifest in the first microarray diagnostic device and application (Roche AmpliChip™) approved for use by the Food and Drug Administration.

These three citations are selected as examples from literature, because they represent the dominant thrust in the prior art toward applications of genomic microarrays for medical diagnostics, prognostic assessments and treatment planning. They also hint at applications of interest for public health and individual screening and surveillance, but they do not emphasize or elaborate on methods, apparatus or logistics for such applications.

Specifically, the Cummings and Relman reference elaborates the technology, but not the reduction to practice, that was used in the Epidemic Outbreak Surveillance (EOS) Program—using both pathogen genome and host gene expression microarrays to screen healthy and ill individuals of a population for the presence of and response to viral and bacterial agents of respiratory infections. Two recent publications from the EOS program highlight these infectious disease screening and surveillance applications as pathogen genome resequencing (Lin, B, et al (2006) Genome Research 16, 527-535) and host response gene expression profiling (Thach, et al (2005) Genes and Immunity 6, 588-595).

Specifically, the Golub et al. reference emphasizes identification of diagnostic signatures of peripheral leukocytes to distinguish two different types of leukemia. This critical test would not be undertaken if there were not already determined to be a clinical presence of disease. In the context of the present invention, a routine screening of the same patient material—peripheral blood would either profile mRNA representing a view of global gene expression in the white blood cells, or a more limited array with likely different genes than identified by Golub et al., in order to provide an initial indication that the patient is either healthy, or in early or advanced stage hematological disease. This approach would be useful in discovery of underlying conditions or predispositions affecting the likely state and future course of the individual patient, and of further use in tracking the progression of the disease, if present, much in the manner suggested for use of such arrays in the infectious disease case, above.

Another recent reference highlights this distinction of using microarrays for screening and surveillance rather than for diagnostics and treatment planning. Sharma et al (Diagenic ASA, Norway; Sharma, et al (2005) Breast Cancer Res 7, 634-644) have described informative gene expression profiles for a set of 37 genes assayed from peripheral blood, these profiles having high predictive value for the presence or absence of early stage breast cancer in the tested individual. The notion is that a rapid and relatively non-invasive, systemic surveillance can provide early warning and justify aggressive, more costly and invasive interventions once probable cause for specific disease is established. The powerful advantage from screening applications in this context is the opportunity for earlier detection and likely more effective initial treatment of the disease, prior to appearance of symptoms that would otherwise trigger investigation. The scenario is clearly one with low a priori likelihood (Bayes (1764) Philosophical Transactions of the Royal Society of London, 53, 370-418) for screening in the general population, greater if other data such as family history suggest predisposition, but in any case the screening or surveillance test offers low risk to most patients with a likelihood of very significant benefit for a small number of screened patients. A negative test result may represent a small benefit of comfort to the greater number of screened patients, based on careful evaluation of the assay's likelihood for false negative results.

Sharma et al also cite Whitney, et al ((2002) Proc Natl Acad Sci USA 100, 1896-1901)—a critical reference in this field for assessment of gene expression profiles of individuals for purposes of either screening or diagnostics. This report describes individuality and variations in gene expression profiles from human blood, as a baseline for identification of signature patterns that may belie general and specific responses to particular diseases.

Other references may be provided to suggest applications of peripheral blood gene expression profiling for screening and surveillance related to other disease stages, including but not limited to neurological and neuro-muscular degenerative diseases; pulmonary, cardiovascular and renal diseases; and occupational or incidental exposures to toxic materials and radioactivity.

Gene expression profiling (phenotype) for screening and surveillance applications is also complemented by unequivocal identification of the genotype as DNA sequence and haplotypes of specific genes that may represent singular and dominant disease risk factors, or be part of complex aggregates of genetic factors that predispose increased risk for specific diseases.

It is readily apparent, also, that effective integration of methods and apparatus for screening and surveillance in medical venues will extend to applications in other fields, including veterinary medicine and agriculture, food chain and environmental quality assessments, industrial material manufacture and quality assurance, public health and epidemiology, homeland security and biodefense.

### SUMMARY OF THE INVENTION

This disclosure describes combination of apparatus and methods to comprise a system for broad use and effective application of analytical genomic microarrays for screening and surveillance. Such applications do not exclude potential uses of the invention for clinical diagnostics and may indeed lead to powerful alternative approaches to multiplex diagnostic analysis.

The disclosure identifies favorable combinations of familiar technologies, which together enable an integrated system for development and implementation of useful genomic microarray tests for screening and surveillance in near real time at points of care (use)

The methodology abandons reliance on typical volumes of peripheral blood samples obtained by phlebotomy, in preference for protocols enabling collection, stabilization, archive, extraction and purification of small volumes as obtained from a finger prick. Recommended processing protocols from such starting material favor preparation of sufficient quantities of RNA or DNA of sufficient quality for subsequent steps of targeted amplification and fluorescent labeling.

A strategy is offered for effective integration of capabilities for genotype and phenotype analysis on the same microarray layout. These gene expression re-sequencing arrays (GXR) are well suited for screening and surveillance applications.

Examples are provided for target medical applications of the invention, while recognizing the potential usefulness of the invention across a broader spectrum of non-medical applications.

The invention proposes to reduce to practice prototype apparatus and methods for implementation as an integrated, turnkey analysis system suitable for on-site, near real time applications at points of care (use).

The integrated prototype apparatus includes a novel fluorescent microarray imaging apparatus and methods for its use.

Accordingly, it is an object of the present invention to provide a method of screening for a biological indicator in a patient comprising:

(a) collecting a biological sample from the patient;

(b) extracting genetic information from the biological sample;

(c) applying said genetic information to a genomic microarray,

wherein the microarray comprises a predefined target gene layout, wherein within said target gene layout comprises (i) multiple selected segments of multiple selected housekeeping genes to serve a baselining function and (ii) multiple selected segments of multiple selected genes specifically associated with and representing a gene profile signature for the biological indicator, wherein each segment comprises a short array of re-sequencing features;

(d) performing a gene expression assay to detect local gene sequence variations in the biological sample as compared to a prototype sequence represented on the microarray; and

(e) determining the absence or presence of the biological indicator in said patient based on the data extracted from the microarray.

It is also an object of the present invention to provide a method of differential diagnosis and detection of a biological indicator in a patient comprising:

(a) collecting a biological sample from the patient;

(b) extracting genetic information from the biological sample;

(c) recovering genetic information from a biological sample obtained from said patient at a time predating said collecting and which was stored so as to preserve the structural integrity of said genetic information;

(c) applying said genetic information obtained in (b) to one assay slide of a genomic microarray which is a duplicate array containing up to and including about 150 to 350 gene targets per assay slide, each in triplicate, and applying said genetic information obtained in (c) to the other assay slide of said genomic microarray,

wherein the microarray comprises a predefined target gene layout, wherein within said target gene layout comprises (i) multiple selected segments of multiple selected housekeeping genes to serve a baselining function and (ii) multiple selected segments of multiple selected genes specifically associated with and representing a gene profile signature for the biological indicator, wherein each segment comprises a short array of re-sequencing features; and

(d) performing a gene expression assay to detect local gene sequence variations in the biological sample by comparing the gene expression profile for the genetic information obtained in (b) to the genetic information obtained in (c).

Within this object, it is advantageous to correlate the difference between said comparing to the absence or presence of a disorder represented by said biological indicator.

Further, it is an object of the present invention for the method of differential diagnosis and detection to be an iterative process. As such, each of (a) and (b) may be repeated after any predetermined time interval measure on the basis hours, days, weeks, months, or even years. In this method, the new genetic information obtained is compared to the t=0 or baseline standard referred to in (c). Subsequently, the microarray detection and comparative analysis is performed. In this case, progression and/or improvement of the underlying etiology of the biological indicator is determined on the basis of the gene expression profile for the selected target genes by a time based comparison of the gene expression profile as compared to the t=0 (or baseline) sample and any preceding sample recovered and anylzed subsequent to the t=0 (or baseline) sample.

The above objects highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following Figures in conjunction with the detailed description below.

FIG. 1 illustrates the work flow for the PAXgene Blood DNA process.

FIG. 2 illustrates the work flow for the PAXgene Blood RNA system.

FIG. 3 is a perspective view of a single Affymetrix cassette format adjacent to 96-well plate formatted array of microarrays.

FIG. 4 shows Whatman FTA paper cards used for RNA/DNA analysis.

FIG. 5 illustrates the GeneReleaser bind-denature-release product from Bioventures, for obtaining multiple assays from a single human blood sample.

FIG. 6 shows the Fuji Medical Systems QuickGene 810c system for rapid preparation of high quality RNA and DNA from blood and tissue specimens.

FIG. 7 shows one strand of re-sequencing format, read 5′ to 3′ left to right, rows top to bottom, indicating central nucleotide T, G, C, A.

FIG. 8 shows a notional 11 member probe pair set for gene expression assay. The actual array elements are not contiguous.

### DETAILED DESCRIPTION OF THE INVENTION

Unless specifically defined, all technical and scientific terms used herein have the same meaning as commonly understood by a skilled artisan in the fields of chemistry, medicinal chemistry, biochemistry, genetics, and cellular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

System requirements for screening and surveillance—Effective application of microarrays for screening and surveillance, as opposed to use specifically as diagnostic devices, will require minimization of operational cost and physical footprint of the integrated system apparatus and methodology, as well as simplification to remove potential barriers of technical complexity—such as limited opportunity for training of operators to rugged application venues requiring robust operational capabilities.

The screening and surveillance rationale typically involves limited a priori likelihood of significant patient benefit through early watch signs or indicators of disease, although there may be great value as well in a positive elimination of one or more specific or general genetic risk factors for the individual. For widespread use in practical settings the screening rationale imposes pragmatic as well as ethical constraints on total cost per assay including depreciating capital infrastructure, staffing and staff training requirements, reagents and supplies, and time commitments of the provider and patient. In non-medical venues this technology has similar, if not amplified economic constraints driven by cost-benefit considerations and potential scale of the screening and surveillance application, even if the complement of ethical considerations and constraints may be less.

Furthermore, an effective microarray system for screening and surveillance requires logistic and system integration through all steps from sample collection and tracking, physical sample inventories and data archives, specimen material processing and quality controls, data acquisition, data analysis and result reporting. What are the costs for interim and long-term storage of patient specimen material? What is the process for acquiring additional specimen material for re-analysis on occasions of assay failure? How are large individual raw data files translated into decision quality information on the individual subject and how is this information consolidated into cohort or community data?

The subject of this invention addresses these constraints and limitations and offers innovated and integrated approaches to their effective management and resolution.

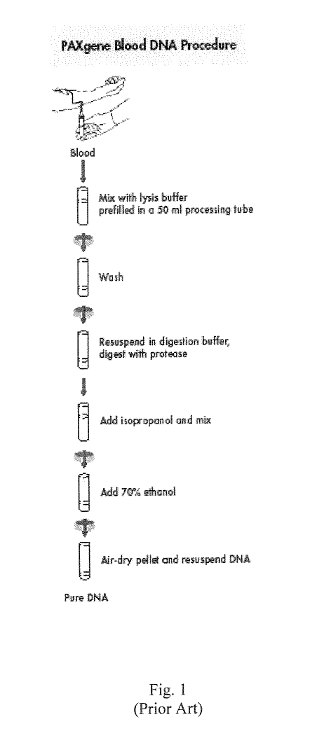

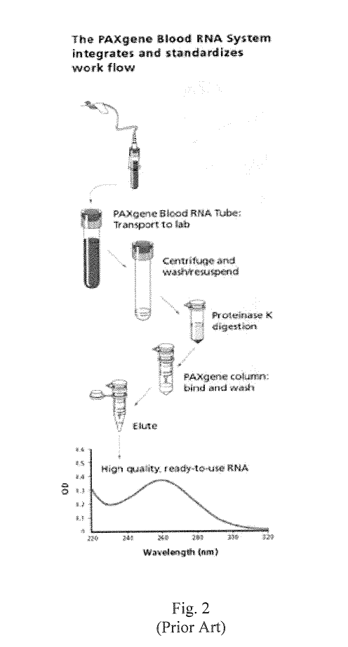

Front-end sample collection, archive, processing and analysis—There is currently an abundance of commercial materials and protocols for extraction and purification of RNA and DNA from biological material for microarray analysis. A particularly favored system is PAXgene (PreAnalytix—a joint venture of BD and Qiagen) for immediate room-temperature stabilization of these sensitive nucleic acid materials until they may be extracted and purified for analysis. Peripheral blood is collected directly into extraction/stabilization media in convenient vials for storage, shipment and first steps in purification. The excellent performance of the PAXgene system is best measured by the reproducible quality of microarray re-sequencing and gene expression profiling data that may be obtained from its DNA (FIG. 1) and RNA (FIG. 2) products, respectively. This accounts for the extensive use of this approach in research applications, and this is amplified by several commercial enterprises offering PAXgene RNA and DNA preparation and high-end Affymetrix photolithographic microarray analysis services (example Expression Analysis, North Carolina). The insets below illustrate the flow of PAXgene collection to DNA and RNA.

We note from standard PAXgene RNA and DNA protocols that both require up-front handling and centrifugation separations for significant sample volumes, 15 ml and 50 ml per sample for RNA and DNA respectively. There then follow multiple precipitations, transfers, resuspensions, incubations and centrifugations of individual samples until final products are ready for PCR or microarray labeling and analysis. This all requires capital laboratory equipment, significant benchspace, and skilled laboratory personnel as expected for a laboratory R&D venue, but not for the limited space and personnel constraints of a clinical or non-medical point of care (use).

Direct experiences of the inventor and colleagues in the EOS Program, and discussions with Expression Analysis, are consistent actual costs for the PAXgene through microarray analysis are on the order of $1,000 to $2,000 per assay, exclusive of costs for sample storage, and the tracking of physical inventories and analysis data.

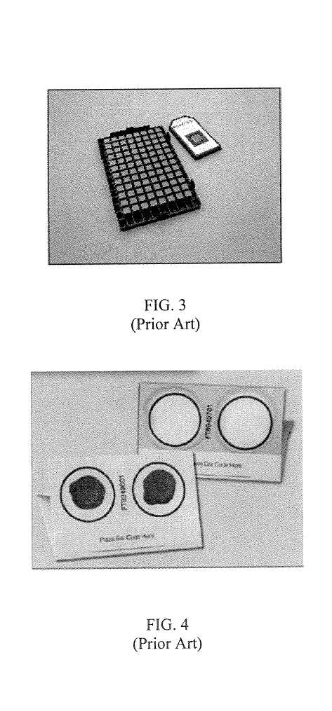

A high throughput analytical gene expression profiling system has been commercialized as six operating components, for analysis of up to 200 to 300 samples per day.
A Qiagen robot for automated parallel processing extracts RNA from up to 96 samples (using microtiter plate formats).
A second Qiagen robot automates parallel sample processing to remove the abundant globin mRNA that can interfere with downstream gene expression analysis
A Caliper robot automates parallel sample processing to amplify purified RNA/DNA, introduce targets for indirect fluorescent labeling,
The labeled products are hybridized to a next generation of Affymetrix microarrays (FIG. 3) that have higher feature density and smaller size, each fitting into the bottom of one well of a 96-well plate. Hybridization and washing of these arrays is performed on the Caliper target preparation robot.
The plates of prepared microarrays are scanned using the ImageExpress 5000 confocal microarray plate scanner (Axon, Molecular Tools)

It is important to note the high aggregate cost to purchase and operate such a system: about $800,000 for capital equipment and about $1,000 per assay

It is also important to note that the system only makes high throughput processing more efficient and it does not decrease the cycle time required to process any individual sample, about 48 hours. It is high-throughput and relatively labor-efficient, requiring only one or two operators and processes 96 samples in the same time required for a single operator to process one or a few samples manually. At this time, the microarrays for research applications with the high throughput system are offered at about the same price as the larger, single analysis microarray formats used in typical microarray analysis facilities.

These high cost estimates represent a barrier to the scale and experimental design of clinical research evaluations safety and efficacy associated with potential products—devices or drugs. Such research and trials would us such microarrays to establish analytical endpoints as recommended in recent FDA Guidelines to the Pharmacogenomics Industry. Such costs also represent a barrier to widespread use in screening and surveillance operations, particularly if there is limited probable cause to justify a screening test for a particular individual and if there is (yet) no commonly accepted path for reimbursement of the cost of such a test.

Part of the incentive leading to the present invention is the notion that a simple turnkey system for use of genome microarray technology at the point of care (point of use) will reduce total costs of operation to a level that will both enable effective screening and surveillance applications, and also favor the design, execution and pricing of more future clinical research and trials.

Therefore, for purposes of this invention, alternative approaches to the PAXgene and other commonly accepted sample handling methodologies are described. These proposed and preferred methodologies have prior art applications particularly in the arenas of forensics and individual identification based on unique genetic fingerprints. Such methodologies have been reported as useful and effective for the analysis of limited numbers of genes using real-time polymerase chain reaction (RT-PCR) analysis. There have been noted suggestions, but no known reported data on the quantity and quality of RNA and DNA products from these alternative methodologies in use with re-sequencing or gene expression microarrays.

Chemically treated papers have demonstrated properties to bind whole blood, other tissues or fluids in such a way that cells are lysed and proteins denatured. The process is so effective that DNA from biological materials so processed is reported stable for genetic/genomic analysis after at least 15 years storage (since method developed) and recent reports suggest that RNA may be similarly stable. As an example of such material we cite Whatman FTA Paper (also referred to as “FTA Cards”; see U.S. Pat. Nos. 5,496,562, 5,756,126, 5,807,527, 5,972,386, and 5,985,327), in various formats with recommended protocols suiting a wide range of RNA- and DNA-based applications. (FIG. 4)

Whatman FTA Cards contain chemicals that lyse cells, denature proteins and protect nucleic acids from nucleases, oxidation and UV damage. FTA Cards rapidly inactivate organisms, including blood-borne pathogens, and prevent the growth of bacteria and other microorganisms. FTA Cards offer the following features and benefits:
Capture nucleic acid in one easy step;
Captured nucleic acid is ready for downstream applications in less than 30 minutes. To recover the nucleic acid, a punch is taken from the FTA Card, washed with FTA Purification Reagent and rinsed with TE-1 buffer. DNA on the washed punch is ready to use in applications such as PCR, RFLP analysis and RT-PCR. Since PCR products remain in solution, the punch can be used for multiple amplifications;
Nucleic acids collected on FTA Cards are stable for years at room temperature;
FTA Cards are stored at room temperature before and after sample application, reducing the need for laboratory freezers;
Suitable for virtually any cell type;
Indicating FTA Cards change colour upon sample application to facilitate handling of colourless samples;
FTA Cards are available in a variety of configurations to meet application requirements; and
The configuration of the FTA Cards may be readily customized.

It is a noteworthy safety consideration that potentially infectious agents in blood or other tissue and fluid are immediately inactivated (though genomically intact) on the FTA paper.

A similar bind-denature-release product and methodology is GeneReleaser (Bioventures, Tennessee). GeneReleaser® is a five-minute protocol that provides PCR Ready DNA/RNA quickly over multiple assays, yielding faster, simpler and more reliable PCR results than other reagents and negates the need for purifying DNA/RNA. GeneReleaser® is composed of proprietary polymeric materials, GeneReleaser® quickly facilitates release of DNA from cells or other materials containing genetic materials in a form suitable for PCR amplification. It also segregates inhibitors released during lysis, along with preservation agents that may interfere with amplification. Further, it consistently provides amplifiable nucleic acids from minute amounts of material, consequently conserving often precious or rare sample materials.

GeneReleaser® achieves lysis, releasing the DNA/RNA from the sample directly in the amplification tube on a thermocycler within minutes; this time frame can be shortened using a microwave protocol. Full protocols for DNA/RNA preparation from diverse types of samples, including blood, sputum, bacteria and tissue cultures, as well as bacteria phages, paraffin embedded tissues, biopsies, mouse tail, plants and for such infectious agents as MTB and HIV are available.

With GeneReleaser®, sample preparation to full PCR readiness occurs within just five minutes. PCR-ready DNA/RNA are economical on a per sample cost basis. PCR-ready DNA for multiple assays can be obtained from a single sample of human blood, 0.2 ml specimens with about 10⁶ nucleated blood cells, using a 6 to 12 minute protocol and a laboratory thermocycler. The company recently announced a new material product configuration suited to direct absorption and rapid wash and release purification of RNA (and DNA) from fingerprick samples of whole blood. This appears to provide a suitable alternative material and protocol to the FTA paper, with properties generally favorable for the constraints of this invention's screening and surveillance applications. (FIG. 5)

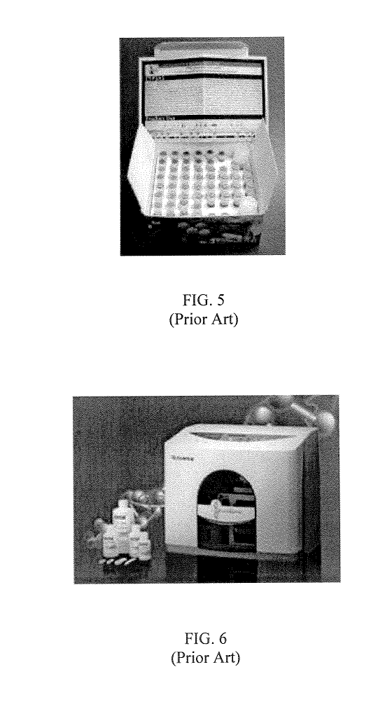

Fuji Medical Systems recently announced its new QuickGene 810c system for rapid preparation of high quality RNA and DNA from blood and tissue specimens, using enclosed and contained 6 to 15 minute protocols and handling from 1 to 8 samples at a time. The proprietary filter membrane frit in the sample processing cell appears to act as the FTA paper, for lysis, binding, washing and selective release of assay-ready RNA or DNA. (FIG. 6)

For the purposes of this invention, these or essentially similar apparatus and methodologies are used in various combinations, optimizing quality and quantity of end-product RNA and DNA for microarray analysis.

The front-end system of the present invention employs such component apparatus and methodology with adaptations and methologies to meet the following requirements as operating and defining benchmarks for the invention:
Material cost less than $30 per sample
   collection material(s)
   storage format
   extraction and purification supplies
Simplest and most common instrumentation
   equipment less than $30,000 (1,000 assays)
   simple microcentrifuge, if required <$5,000
   thermal cycler suited to multiple tasks <$5,000
      sample extraction (per GeneReleaser)
      downstream target amplification and labeling
      duty as fixed temperature incubator as required
   sample processing engine (per QuickGene)
Preparation less than 60 minutes/sample
Less than 60 minutes to process up to 8 samples
Prolonged archival stability for RNA or DNA
Minimal storage requirements
   dry, room temperature
   individually sealed containers
Enabling repetitive assays
   backup for assay failure
   retrospective re-assay
Safe handling
   minimized sample-sample crosstalk
   inactivation of infectious agents
   no hazardous reagents

These requirements are met within an integrated system that successfully translates the physical RNA or DNA product from above through the labeling, data acquisition imaging and data reduction analysis.

Incidental, longitudinal tracking, and automated archival review—A simplified approach of collecting blood or other sample material onto FTA cards or comparable material is described above. This facilitates collection and archive of replicate samples for multiple assays at once or distributed over time. This is important in that it is not necessary to reengage with the individual from whom the sample was obtained in order to repeat an assay in the event of a failed test. This approach also offers opportunities to track samples from the same individual over intervals of time to longitudinally track changes in gene expression phenotype. It is noted that gene re-sequencing results relating to genotype are not generally time variant and single assays would suffice, however one may anticipate in cancer, for example, that biopsy from detected neoplasm may reveal mutation(s) of specific genes established earlier or from normal tissue sampling.

The prolonged stability of samples archived in this manner also affords opportunity for retrospective analysis prompted by research progress and reports of other genes that may be relevant to the ongoing concern or care for the individual. Another segment of the archived sample is excised for assay on a microarray with updated content, or using a future analysis technology not yet anticipated or invented that may offer sufficiently improved sensitivity or specificity or data quality to justify the re-assay.

Part of this invention thus requires an effective physical sample archive and retrieval system as well as a longitudinal data tracking system, as provided by a suitably deployed laboratory information management system (LIMS).

An example of the importance of the LIMS system for data tracking is the opportunity to retrospectively analyze earlier results based on the continuing progress of research and trials appearing in the relevant scientific literature. Specifically, if a global gene expression assay were performed (as Affymetrix U133 2.0 Plus), results may be analyzed for signatures prompted by blind analysis of the data or by results of studies published in the current literature. Later studies may offer insight and informatic value from previously unknown or unappreciated signature elements. Archives of raw assay results should be readily accessible for updating reviews.

Microarray content for analysis of genotype and phenotype—The applications and purposes of this invention are well served by designing of microarray content to enable simultaneous assessment of genotype and phenotype. Using the example of Affymetrix photolithographic microarrays as commercially available products today, the content layouts are different for assays of genotype (CustomSeq re-sequencing) and gene expression phenotype (U133 2.0 Plus).

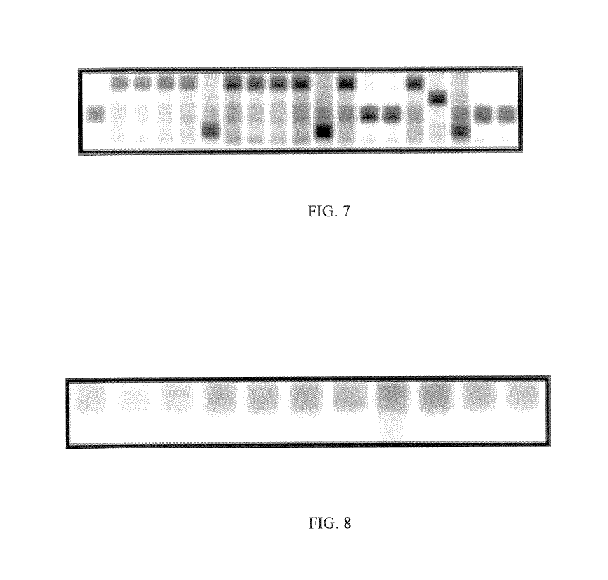

A re-sequencing rationale for genotyping microarrays (reference Cutler, et al (2001) Genome Res 11, 1913-1925; see also US 2006/0210967) offers multiple contiguous features to test the local sequence at each consecutive nucleotide position—the test including the expected nucleotide and the three alternatives, and also the four corresponding nucleotides of the complementary DNA strand. Such sets of eight features per base may extend dozens to thousands of nucleotides of sequence from selected genes. FIG. 7 shows one strand of re-sequencing format, read 5′ to 3′ left to right, rows top to bottom, indicating central nucleotide T, G, C, A.

This system offers significant advantage of unequivocal results for both detection of and specification of particular gene segments—with consequence of remarkably minute likelihood of false positive results based microarray performance. False positive results may result from upstream crosstalk between multiple samples or operator errors in sample collection and handling.

Alternatively, gene expression microarrays (Affymetrix and other formats) typically employ one or more presumably unique oligonucleotide probes to indicate interaction with mRNA-derived target sequences. The natural, sequence-dependent variations in hybridization kinetics and hybrid stability over short distances (25 nucleotides for example) and the natural, often confounding sequence similarities of different genes over short distances—can either compromise detection altogether (as with non-specific interference by abundant globin mRNA from peripheral blood) or lead to specific but unanticipated false positive results.

The Affymetrix U133 content is designed to represent each gene with about 11 different sampled sequences (as 25-mer oligonucleotide probes) across the 3′ terminal sequence (about 600 nucleotides) of the gene's estimated or established mRNA sequence. Each of the 11 probes is complemented with a control feature bearing a deliberate nucleotide mismatch at the center nucleotide position. This system is well controlled, but certainly imperfect across the spectrum of all possible test results. FIG. 8 shows a notional 11 member probe pair set for gene expression assay. The actual array elements are not contiguous.

A key feature of the present invention is the design of microarray contents to represent multiple selected segments of multiple selected genes, each segment as a short array of re-sequencing features. This layout of content enables a gene expression assay to be performed and quantitated, using sums of relative fluorescence intensities at the indicated nucleotides re-sequencing representations of gene segments. It is clear that this approach simultaneously enables detection of local gene sequence variations of the sample target material from the prototype sequence represented on the microarray. This combination of content layout, assay and analysis may be called gene expression by re-sequencing (GXR).

One extension of this approach is envisioned using preparations of DNA and RNA from separate aliquots of the same sample, using distinct fluorescent labels for each. DNA sequences of gene segments would be assessed from one label of the mixture, leveraging the one or two copies of unique gene sequence per cell, as the natural distribution of signal intensities across the features of the array that indicate that gene segment's DNA sequence. Relative amounts of mRNA representing gene expression would be assessed from variations of signal of the second fluorescent label, as integrated sums of intensities across the same features of sampled sequence.

The design of microarray content for GXR analysis as envisioned by this invention requires the combined assessments of genome information from multiple public databases and other resources—the particular combinations of resources and their application in ultimate design of microarrays is not particularly obvious.

The primary selection of genes and gene segments to be represented must offer value to the end-user in the context of screening, surveillance and other applications. If individuals are to be assessed with respect to particular disease risk factors, then the GXR microarray should offer a multiplicity of individual tests that might typically be offered alone or together using contemporary best practice methodologies.

By way of example, this aspect of the present invention is described with reference to applications related to a small subset of etiologies of diabetes mellitus, specifically known as Mature Onset Diabetes of the Young (MODY). However, in no way is this example intended to be nor is limiting upon the application of the scope of disorders to which the present invention applies. For this example, thus far six different autosomal dominant alleles of genes have been identified as etiologic in the onset of non-insulin dependent type II diabetes in young adults. Once an individual is diagnosed with MODY, management of the disease may be optimized based in part on knowledge of which gene is at cause (diagnostic application).

Greater benefit will likely be derived from applications of the invention for siblings and offspring (screening application), as this group bears greater a priori risk for MODY (probable cause for significant value from test results). The test offers opportunity for longitudinal surveillance and earlier recognition of disease onset, and possibly pro-active pre-symptomatic intervention to delay onset or mitigate consequences of delayed recognition of disease onset.

Online Mendelian Inheritance in Man (OMIM) is a public database resource (http://http://www.ncbi.nlm.nih.gov/entrez/query.fcgi?db=OMIM) linked to GeneTests, an NIH-supported database resource operated by the University of Washington (http://www.genetests.org/). Under GeneTests' reviews for diabetes the six MODY instances are referenced among 16 broad categories of genetically linked diabetic disorders. Several international clinical and research facilities offer DNA sequencing across coding sequences of the implicated genes to determine if a particular individual bears the causal allele.

| MODY Type | Target Gene | Allelic Determinants | Test Facilities |
|---|---|---|---|
| I | HNF4A | 6 | 3 |
| II | GCK | 10 | 6 |
| III | TCF1 | 21 | 7 |
| IV | IPF1 | 9 | 3 |
| V | TCF2 | 7 | 5 |
| VI | NEUROD1 | 2 | 1 |

Using GenBank as the public resource database for reference human genome sequences, gene segments including and flanking these 54 (known) allele variations related to MODY could all be tiled as part of the content of a single assay microarray. Assuming use of 50 contiguous nucleotides of sequence across each allele variant, and 8 features per nucleotide, this composite assay requires only 21,600 features—on perhaps a 150×150 feature microarray. Affymetrix manufactures custom layouts on its CustomSeq™ re-sequencing microarrays as approximately 500×500 layouts of 20μ×25μ features. Such an array can clearly accommodate these known MODY analyte sequences, and in addition another 500 to 600 allele variants that might represent other direct and indirect factors in the more abundant class of type II diabetes. Ultimately the usefulness of such an informatically rich screening, surveillance (and diagnostic) capability will be determined, and issues of cost and peformance of the assay system of the present invention will facilitate such testing and determinations.

DNA from a fingerprick sample of peripheral blood, or exfoliated cells on a buccal swab would be assayed in this invention to evaluate the individual's DNA sequence at all known candidate MODY alleles. The projected cost for such an aggregate microarray assay would certainly be less than the cost for sequencing the coding region(s) of any one of the six known causal genes.

Again, it should be noted that the description above for MODY is merely illustrative of the present invention. As such, the DNA sources identified for MODY analysis also apply to the larger spectrum of disorders to which the present invention applies. More specifically, the DNA (also referred to as “nucleic acid”) for use in the present invention may be obtained by collecting a biological sample from a patient and subsequently directly applying the sample to further microarray analysis or extracting (i.e., isolating or substantially purifying) the DNA from the sample for further analysis. In this regard, the biological sample includes, but are not limited to, peripheral blood; exfoliated cells, including cells obtained from a buccal swab, stool, sputum, nasal wash, nasal aspirate, nasal swab, throat swab, vaginal swab, and rectal swab; blood, blood cells (e.g., white cells), tissue or fine needle biopsy samples, urine, peritoneal fluid, visceral fluid, and pleural fluid, or cells therefrom.

In the context of the present invention the term “genetic information” refers to DNA, including cDNA, or RNA, including mRNA and rRNA.

Further, the present invention is not limited to biological samples obtained from humans. The present invention may also be applied to biological samples obtained from any animal species including domestic and/or farm animals including, but not limited to: dogs, cats, horses, cows, pigs, goats, sheep, rabbits, mice, rats, etc. In addition, the present invention may also be applied to biological samples obtained from any animal species that may be found in the wild or traditionally thought of as zoological animals, for example: monkeys, giraffes, elephants, zebras, tigers, lions, lemurs, etc. Further, the present invention may also be applied to biological samples obtained from any avian species. In this embodiment it is understood that the gene targets embedded on the microarray chip to be detected would contain the genes for the respective species selected.

Further, the sample of the present invention is not limited to biological samples, the sample of the present invention may be environmental (air, water, soil, etc.), animal (see above), or plant (e.g., cells obtained from any portion of a plant where the species of plant is without limit). Again, in this embodiment it is understood that the gene targets embedded on the microarray chip to be detected would contain the genes for the respective species selected, when that species is known (i.e., animal or plant). Further, when the sample is environmental, the gene targets embedded on the microarray chip can be any predetermined collection that is used to detect and identify any pathogen of interest, for example.

The microarray assay as enabled by this invention is envisioned to be performed by transfer of a stable FTA paper card of sample(s) to a local service provider, or alternatively in more nearly real time on-site at the point of patient-provider engagement. In the latter case, the far broader range of potential applications for the technology (other non-MODY diabetes genetic factors and other diseases than diabetes) would help justify the initial costs associated with on-site installation and operation of a system defined as the present invention.

As another example of microarray layout design and application under this invention is based on the report of Sharma et al for peripheral blood gene expression profiles related to breast cancer screening. However, in no way is this example intended to be nor is limiting upon the application of the scope of disorders to which the present invention applies. For this example, known allelic variants for each of the 37 informative genes may be identified in current versions of the single nucleotide polymorphism (Entrez-SNP) database resource of the National Center for Biological Information (http://www.ncbi.nlm.nih.gov).

The table below shows the current numbers of allele variants for 11 of the 12 genes of the profile set having the highest predictive scores among patients found to have breast cancer or having no signs of breast cancer. Using 50 nucleotides per variant allele, this set of 544 allele/SNP targets can be readily accommodated on a single GXR custom designed screening microarray layout.

| | | Entrez-SNP |
|---|---|---|
| Accession Number | Gene | Human Entries |
| BC000514 | RPL13A | 25 |
| BC007512 | RPL18a | 12 |
| BC019093 | GNB2L1 | 72 |
| BC009696 | IFITM2 | 63 |
| BC047681 | S100A9 | 19 |
| BC066901 | H3F3A | 20 |
| BC034149 | RPS3 | 70 |
| BC047681 | S100A9 | 19 |
| BC001126 | RPS14 | 36 |
| NM_000980 | RPL18A | 12 |
| AY495316 | COX1 | 196 |

The number of allele/SNPs selected may be reduced if available data suggests specific loci are located outside of coding sequences or a given gene's exon sequences. As experience and literature reports accumulate for this system, iterated updates of the microarray layout are anticipated.

Although there is limited if any specific information at this time on correlation of specific alleles in this set with breast cancer phenotypes, the GXR assay has the advantages. First and foremost is an immediate low cost implementation for screening purposes, and the longer term accrual of results that will bear on specific alleles of each gene as a predisposing factor for breast cancer.

Furthermore, the representation of each of the genes as multiple allelic sequence segments offers a level of quantitative redundancy and corroboration for estimations of relative levels of gene expression across the profile of 37 genes.

Selected short sequence segments of these variant alleles can be represented on a breast cancer screening microarray. Using labeled RNA preparations from peripheral blood samples, the relative gene expression profiles of these genes may be determined on the microarray, summing fluorescence signal intensities across respective GXR sequence representations. The gene expression profile and the individual's complement of genotypes for these probe genes are revealed in the same assay. It should be noted that the representations of each allele would likely be that sequence of the most commonly occurring allele in sampled populations. The re-sequencing layout enables specification of multiple alleles at each locus with a single prototype sequence representing that locus on the microarray.

Amplification and labeling considerations—A constraining aspect of the current invention is the preferred embodiment using small sample volumes from samples obtained by minimally or non-invasive methodologies (buccal swab, fingerprick blood, nasal wash, etc.). A single Affymetrix U133 gene expression assay typically uses all of the RNA extracted from one or two 2.5 ml PAXgene extraction kits. This invention proposes useful assays with at least 10-fold smaller volumes of peripheral blood, and correspondingly smaller yields of nucleated cell DNA and RNA. Extensive experience of prior art indicates that the approximately 106 nucleated cells per 0.2 ml peripheral human blood is ample for DNA preparations and PCR-based amplification and sequencing of specific gene targets. Similar experience favors real-time PCR assays of specific gene expression following initial reverse transcription of mRNA preparations from small volumes of blood.

The reduction of this invention to practice emphasizes the need to employ any of various multiplex amplification methods to provide sufficient labeled target material for effective hybridization and imaging on fluorescent microarrays.

As selected sets of target genes are identified for a screening or surveillance application, the respective sequences of each gene in the set are identified in the GenBank reference database. The particular subset sequences flanking known allelic variants are specified by reference to the Entrez-SNP reference database. From this information, common oligonucleotide primer selection tools will be used to identify candidate primers for amplification of target regions in multiplex PCR cocktails. Proposed amplicons of several hundreds of basepairs can be selected which both improves the likelihood for identification of favorable primers (uniqueness, base composition, sequence), and will likely provide amplicon products that span multiple loci of variant alleles.

Validated combinations of multiplex primer cocktails for use with specific GXR microarrays will represent patentable claims as intellectual property in their own right.

As the bioinformatics survey of target gene sequences and allelic variant loci progresses, a systematic search of the pooled sequences will likely reveal specific restriction enzyme sites that are absent from and favorably flank in proximity the target amplicon sequences. Various methods are familiar in prior art to attach unique primer oligonucleotide sequences to the ends of such restriction fragments.

In this manner, a general amplification of the genomic restriction fragments can provide sufficient material to illuminate the represented sets of genes on this invention's GXR microarray formats.

Alternatively, methodologies have been established for random or generic amplification of DNA sequences by replicative polymerases, and such products have been favorably assessed for specific interactions with re-sequencing microarray formats.

It is noted from experience and prior art that some classes of total RNA or total genomic DNA may interfere with high density microarray analysis of genotype or phenotype. Abundant and interfering globin mRNA has been successfully removed from preparations by hybridization to sequences complementary to the 3′ end of globin transcripts attached to paramagnetic beads. Abundant and interfering repetitive sequences in genomic DNA have been successfully removed from preparations by hybridization blocking with the Cot 1 fraction of purified human cell culture DNA (Roche).

In the context of the present invention such methodological interventions represent processing complexity and potential delays mitigating against a more nearly real-time analysis on-site. The proposed application prefers design and execution of specific multiplex amplification strategies using RNA or DNA preparations from small sample volumes, focused on limited subsets of the entire genome.

Products of multiplex amplification will be assayed on commercially available whole genome formats to validate the success and representative presence of target gene sequences.

The labeling of products of multiplex amplification is accomplished by various methods, alone or in favorable combination. Specifically the amplification reactions themselves may include fluorescently labeled substrate nucleotides, or alternatively biotin-labeled nucleotides that favor post reaction processing with fluorescently labeled (phycoerythrein) streptavidin and immunoamplification with crosslinking antibodies to streptavidin. Alternatively practice may determine that fluorescent primers used in multiplex amplifications may provide sufficient levels of amplicon labeling for quantitative measure of interactions with probes immobilized on the microarray.

By way of example, qRT-PCR amplification/labeling may be used, wherein one or more oligonucleotide primer pairs will be identified for specific amplication and initial biotin-labeling of specific gene transcripts represented in the total RNA preparations. The deliberately simple strategy employs a single reaction tube for each sample. The first-strand cDNA synthesis cycle with reverse transcriptase extends from the gene transcripts' 3′-proximal primer(s). Second and subsequent thermal cycles incubate at elevated temperature for Taq DNA polymerase chain reaction using both gene-specific primers. cDNA and DNA amplicon products bear biotin labels from incorporation of deoxyribonucleotide mixes containing biotinylated dCTP and/or biotinylated dATP, along with the standard dATP, dCTP, dGTP and dTTP.

The course of amplification/labeling reactions will be monitored by qRT-real time PCR. In a single reaction tubes, first-strand cDNA synthesis is performed with the starting total RNA preparation, reverse transcriptase (Invitrogen SuperScript™ III) and the primer complementary and 3′ proximal to the target gene's mRNA sequence. Subsequent cycles of cDNA amplification use Taq DNA polymerase (Invitrogen Platinum®) and both amplification primers. The detection method is based on enhanced fluorescence of SYBR® Green fluorescence binding to the accumulating duplex cDNA through successive amplification cycles.

Since this exemplary method utilizes only the single SYBR® label, limiting distinction of multiple products in multiplex reactions for amplification and labeling of multiple gene targets. More elaborate multiple fluorescent-primer conjugate labels for RT-PCR are available, but prohibitively expensive to develop and apply for the genes of interest in this Phase I project. To demonstrate respective amplicon products of different target genes based on amplicon product lengths, as predicted by primers and target gene cDNA sequences the Agilent BioAnalyzer 2100 system (at CBMSE-NRL) with DNA electrophoresis chips may be utilized.

Design of qRT-RTPCR primers for multiplex labeling amplifications is based on 3′-proximal coding and untranslated sequences of each gene's mRNA sequence, including post-transcriptional RNA splice junctions. The nucleic acid sequence for each of the target genes of interest can be accessed through NCBI's Entrez Gene database link to GenBank, and mRNA structure is mapped and linked to annotation of the mRNA/cDNA sequence.

It is highly preferred that target gene amplicon sequences substantially overlap with the sequences of DualChip™ Xmer probes on the microarray, in order to hybridize specifically as labeled targets with the probe DNA sequences on the Eppendorf DualChip™microarrays. The DualChip™ Xmer probes are sequences of 200 to 400 nucleotides representing the 3′-proximal sequence of each probed gene's mRNA sequence. Therefore primers for target gene-specific qRT-PCR amplification of total RNA will be selected under constraints of the 3′ proximal coding and untranslated mRNA sequences and 3′-polyadenylate, noting that primers from this region must also be screened for unique sequence and sequence-dependent physical-chemical properties in order to work well in multiplex analysis of the genes of interest.

Several primer oligonucleotide design software packages are available, and most include considerations for design of multiplex primers to co-amplify sequences of multiple but specific target genes. Examples include Primer3, Primo (and variants), PrimOU—and have used these to design whole viral genome re-sequencing primers as well as multiplex PCR primers for detection and identification of multiple (dozens) of pathogens and strains on re-sequencing microarrays.

Important empirical and general constraints on primer design to favor successful multiplex applications are summarized in Qiagen product literature—prescribing
Oligonucleotides that are 21 to 30 nucleotide length with unique sequence signature by BLAST
GC content in range 40 to 60%
Annealing temperature 4° C. to 8° C. less than lowest primer Tm
Avoid complementarity 2 to 4 bases at 3′ ends to reduce primer-dimer formation
Avoid mismatches at 3′-end of primer and target-template sequence
Avoid runs of 3 or more G and/or C at 3′ end of primer
Avoid complementarity of sequence within and between primers.

Microarray content for selected applications—Buccal swabs or fingerprick blood samples will certainly provide sufficient DNA for PCR-based amplifications and determinations of particular target genotypes. The peripheral blood samples are preferred for practice of screening and surveillance of immune system responses to early or advanced disease states, even in the absence of overt symptoms and complaints. However, any of the samples identified herein above may be used in the present invention.

Increasingly the NCBI-NIH Gene Expression Omnibus (GEO) is a useful resource database for examples of differential gene expression analyses for specified health and disease states, featuring microarray results representing the broad variety of commercial and proprietary local analysis platforms.

In the context of this invention for application of genomic microarrays in screening and surveillance, several favorable examples are cited where informative sets of gene expression profiles from peripheral blood have been identified in the literature.

(a) Cancer -

| | | |
|---|---|---|
| (i) | Breast Cancer | Sharma et al (2005) |
| (ii) | Colon Cancer | Solmi et al (2004) and Solmi et al (2006) |
| (iii) | Lung Cancer | Kaminski and Krupsky (2004) |

(b) Neuromuscular and neurodegenerative disease

| | | |
|---|---|---|
| (i) | Multiple Sclerosis | Achiron et al (2004) |
| (ii) | Alzheimers Disease | Diagenic ASA |
| (iii) | Muscular Dystrophy | |

(c) Response to occupational and incidental exposures

| | | |
|---|---|---|
| (i) | Radiation | Amundson et al (2004) |
| (ii) | Metal Fumes | Wang et al (2005) |
| (iii) | Benzene | Forrest et al (2005) |
| (iv) | Tobacco | Lampe et al (2004) |

(d) Cardiovascular disease and stroke

| | | |
|---|---|---|
| (i) | Stroke | Moore et al (2005) |
| (ii) | Heart Failure | Seiler et al (2004) |
| (iii) | Hypertension | Bull et al (2004) |

(e) Infectious agents and organ transplantation

| | | |
|---|---|---|
| (i) | Liver | Green and Michaels (1992) |
| (ii) | Kidney | Sinert and Erogul (2006) |

In no way is the present invention and the scope of applications limited to the gene targets and/or disorders indicated above. The present invention is amenable to and embraces any disorder whether human or non-human animal based, any pathogen, any condition (e.g., exercise related expression changes, irradiation-mediated expression changes, etc.), etc. Genes for identifying the root of and/or tracking the progress of the foregoing can be selected from public databases and are at the discretion of the artisan.

The present invention identifies three examples of applications of simple sets of host gene expression targets to be analyzed by multiplex RT-PCR amplification from mRNA preparations. Typically these sets would represent on the order of a dozen or so familiar housekeeping gene targets that do not tend to vary (individually) from specimen to specimen across a wide variety of donor physiological states. These provide a baseline pattern of gene expression level for the individual sample, as a group reducing likelihood of radical variation from circumstances that might affect one or two single genes of the baseline set.

Because of availability on the commercial gene expression microarrays proposed for research and development (Eppendorf DualChip) and because of commericially available primers for their individual RT-PCR reactions, we propose to develop and optimize multiplex RT-PCR cocktails that will reliably amplify and provide a reproducible pattern of gene expression levels (amplified RNA yields) across the set of baseline genes.

We elect an RNA amplification strategy because our preferred sample is preferably a dried, preserved blood stain prepared on Whatman FTA™ paper. The volume of initial blood represented in a punch taken from the FTA card is likely to represent an aggregate of only several thousand to several tens of thousands of white blood cells as source of mRNA for profiling. Use of the FTA cards, or equivalent methodology for sampling, preserving and providing small volumes of blood for immediate and deferred analysis is a preferred part of the overall process.

The Eppendorf DualChip microarrays provide duplicate arrays containing up to and including about 150 to 350 gene targets per assay slide. However, the microarray could be tailored to suit the needs of the practitioner of the present invention to accommodate lower numbers of gene targets. These arrays of genes include the 13 or so standard housekeeping gene set referenced above. The test genes on the arrays are selected for particular application domains, including sets related to states of human aging, apoptosis, cancer (a general set and a specific breast cancer set), inflammation, side effects of small interfering RNA. On each of the duplicate arrays, each gene target is represented in triplicate (thus about 1000 features or target spots per array).

The present invention demonstrates the approach and methods for three applications, each having particular roles for inflammation responses. These include:

samples from control patients, and others with optic neuritis that may or may not receive drug for delay/mitigation of active or possible future multiple sclerosis

samples from individuals prior to (control) and immediately after (test) periods of intense exertion/exercise

samples from individuals prior to (control) and immediately after (test) exposure to ionizing radiation for diagnostic imaging.

By way of example, the table below identifies the housekeeping baseline gene profile set and experimental gene sets for each of the three studies above, noting that part of the selection is based on reports in the literature and part based on the available population of gene targets on the Eppendorf Inflammation DualChip.

Candidate Human Genes of Interest:

| | |
|---|---|
| Housekeeping genes | ACTB, ALDOA, GAPD, HK1, HPRT1, |
| | K-ALPHA-1, MDH1, PPIE, RPL13A, RES9, |
| | SDS, TFRC, PLA2 |
| Ionizing radiation genes | CCR1, CDKN1A, CX3CR1, FOS, |
| | IL13, IL8, SELL, STAT1, TNF, TNFSF10 |
| Acute exercise genes | CCL5, EGR1, FOS, H1F1A, IL1RN, IL2RB, |
| | IL6, JUN, MYC, NCAM1, PDGBRF, STAT4 |
| Multiple sclerosis genes | CD28, IL1B, IL1RN, ITGG2, MAP3K1, |
| | MAP2K8, MAPK14,-MAPK9, NFKB1, |
| | TGFB1, TNF, TNFRSF1B, TNFSF14, TRAF1, |
| | TRAR6 |

The acronym names of the genes in the lists above, and links to their sequence and functions are readily decoded by search engine query or directly through the NCBI-NLM-NIH Gene Database interface
http://www.ncbi.nlm nih.gov/entrez/query.fcgi?CMD=search&DB=gene

Given the small size of these example gene target sets and baseline sets of genes, the resulting data from surveys of affected and control samples will be analyzed using a neural network classifier, as vectors of combined housekeeping and test gene expression levels mapped to categories such as before/after or treated/untreated or affected/control. Training of the neural network for this application will be supervised. Subsets of the total available data will be jackknifed (not included) from training for quantitative assessment of the trained network performance. Performance is assessed as likelihood of network prediction of correct sample category from individual data vectors.

Experinentally, the collected data sets will also be evaluated through unsupervised training algorithms (self organizing map) and the performance of trained networks similarly assessed.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Numerous modifications and variations on the present invention are possible in light of the above teachings. It is, therefore, to be understood that within the scope of the accompanying claims, the invention may be practiced otherwise than as specifically described herein.

### REFERENCES

1. Achiron, A, Gurevich, M, Friedman, N, Kaminski, N and Mandel, M (2004) Blood transcriptional signatures of multiple sclerosis: unique gene expression of disease activity.
2. Affymetrix Technical Note (2003) Globin reduction protocol: a method for processing whole blood RNA samples for improved array results.
3. Affymetrix Technical Note (2004) GeneChip® expression platform: comparison, evolution, performance.
4. Amundson, S A, Grace M B, McLeland, C B, Epperly, M W, Yeager, A, Zhan, Q, Greenberger, J S and Fornace, A J, Jr (2004) Human in vivo radiation-induced biomarkers: gene expression changes in radiotherapy patients.
5. Baranzini, S E, Mousavi, P, Rio, J, Caillier, S J, Stillman, A, Villoslada, P, Wyatt, M M, Comabella, M, Greller, L D, Somogyi, R, Montalban, X and Oksenberg, J R. (2004) Transcription-based prediction of response to IFNb using supervised computational methods. PLOS Biol 3, e2.
6. Bertholet, V and de Longueville, F (2004) Gene expression profiling with DualChip™ microarrays. Eppendorf Application Note No. 79, pp 1-8.
7. Bomprezzi, R, Ringner, M, Kim S, Bittner, M L, Khan, J, Chen, Y, Elkahloun, A, Yu, A, Bielekova, B, Meltzer, P S, Martin, R, McFarland, H F and Trent, J M. (2003) Gene expression profile in multiple sclerosis patients and healthy controls: identifying pathways relevant to disease. Hum Mol Genetics 12, 2191-2199.
8. Bull, T M, Coldren, C D, Moore, M, Sotto-Santiago, S M, Pharn, D V, Nana-Slinkam, S P, Voelkel, N F and Geraci, M W (2004) Gene microarray analysis of peripheral blood cells in pulmonary arterial hypertension. Am J Resp Crit Care Med 170, 911-919.
9. Collins, F S (2005) Personalized medicine: How the human genome era will usher in a health care revolution. Presentation to the Personalized Medicine Consortium, 10 Feb. 2005.
10. Collins, F S, Green, E D, Guttmacher, A E and Guyer, M S (2003) A vision for the future of genomics research. Nature 422, 835-847.
11. Connolly, P H, Caiozzo, V J, Zaldivar, F, Nemet, D, Larson, J, Hung, S, Heck, J D, Hatfield, G W and Cooper, D M. (2004) Effects of exercise on gene expression in human peripheral blood mononuclear cells. J Appl Physio 97, 1461-1469.
12. de Longueville, F (2004) Xmer technology: probe selection for DualChip™ microarrays. Eppendorf Application Note No. 78. pp 1-4.
13. de Longueville, F, Chapelier, M and Boffe, S. (2004) Expression profiling on a low-density DNA microarray. Eppendorf Application Note No. 71, pp 1-4.
14. de Longueville, F, Chapelier, M and Boffe, S. (2004) Expression profiling on a low-density DNA microarray. Eppendorf Application Note No. 71, pp 1-4.
15. Del Rio, S (2001) Cost-effectiveness in sample processing using the FTA™ treated stain card for high throughput. Poster from Promega 8th International Symposium on Human Identification.
16. Eisen, M B, Spellman, P T, Brown, P O and Botstein, D. (1998) Cluster analysis and display of genome-wide expression patterns. Proc Natl Acad Sci, USA 95, 14863-14868.
17. Evans, W E and McLeod, H L (2005) Pharmacogenomics—disposition, drug targets and side effects. N Engl J Med 348, 538-549.
18. Finn, O J (2005) Immune response as a biomarker for cancer detection and a lot more. N Engl J Med353, 1288-1290.
19. Forrest, M S, Lan Q, Hubbard, A E, Zhang, L, Vermeulen, R, Zhao, X, Li, G, Wu, Y-Y, Shen, M, Yin, S, Channock, S j, Rothman, N and Smith, M T (2005) Discovery of novel biomarkers by microarray analysis of peripheral blood mononuclear cell gene expression in benzene-exposed workers. Environ Health Perspect 113, 801-807.
20. FujiFilm Life Science Product Guide. (2005) Nucleic acid isolation system QuickGene-810
21. FujiFilm Life Science QuickGene Application Guide No. 3. (20054) Total RNA isolation from human cultured cell, HEK293, QuickGene RNA cultured cell Kit 8.
22. Grace, M B, McLeland, C B, Gagliardi, S J, Smith, J M Jackson, W E, III and Blakely, W F (2003) Development and assessment of a quantitative reverse transcription-PCR assay for simultaneous measurement of four amplicons. Clinical Chemistry 49, 1467-1475.
23. Green M, Michaels M. (1992) Infectious complications after solid-organ transplantation. Adv Pediatr Infect Dis; 7:181-204.
24. Gronenfelder B and Winzeler, E A (2002) Treasures and traps in genome-wide data sets: case examples from yeast. Nature Reviews (Genetics) 3, 653-661.
25. Hoffman, J (2005) Awash in information, patients face a lonely, uncertain road. New York Times 14 Aug. 2005.
26. International Human Genome Sequencing Consortium (2001) Initial sequencing and analysis of the human genome. Nature 409, 860-921.
27. Jen, K-Y, and Cheung, V G. (2003) Transcriptional response of lymphoblastoid cells to ionizing radiation. Genome Research 13: 2092-2100.
28. Jison, M L, Munson, P J, Barb, J J, Suffrendini, A F, Talwar, S, Logun, C, Raghavachari, N, Belgel, J H, Shelhamer, J H, Danner, R L and Gladwin, M T (2004) Blood mononuclear cell gene expression profiles characterize the oxidant, hemolytic and inflammatory stress of sickle cell disease. Blood 104, 2700-280.
29. Kaminski, N and Achiron, A (2005) Can blood gene expression predict which patients with multiple sclerosis will respond to interferon? PLOS Medicine, Vol 2, e33.
30. Kephart, D and Shenoi, H (1998) Molecular diagnostics: isolation and analysis of RNA from human blood. Promega Notes 68, 23-29.
31. Lampe, J W, Stepaniants, S B, Mao, M, Radich, J P, Dai, H, Linsley, P S, Friend, S H and Potter J D (2003) Signatures of environmental exposures using peripheral leukocyte gene expression: tobacco smoke. Cancer Epidemiol, Biomarkers Prev 13, 445-453.
32. Lander, E S (1999) Array of hope. Nature Genetics 21, 3-4.
33. Lipshutz, R J, Fodor, S P A, Gingeras, T R and Lockhart, D J (1999) High density synthetic oligonucleotide arrays. Nature Genetics 21, 20-24.
34. Lock, C, Hermans, G, Pedotti, R, Brendolan, A, Schadt, E, Garren, H, Langer-Gould, A, Strober, S, Cannella B, Allard, J, Klonowski, P, Austin, A, Lad, N, Kaminski, N, Galli, S J, Oksenberg, J R, Raine, C S Heller, R and Steinman, L (2002) Gene-microarray analysis of multiple sclerosis lesions yields new targets validated in autoimmune encephalomyelitis. Nature Medicine 8, 500-508.
35. Lombardi, S (2004) Industrializing microarrays. Modern Drug Discovery (December 04) pp 46-48.
36. Lombardi, S (2004) Industrializing microarrays. Modern Drug Discovery (December 04) pp 46-48.
37. Mandel, M, Gurevich, M, Pauzner, R, Kaminski, N and Achiron, A. (2004) Autoimmunity gene expression portrait: specific signature that intersects or differentiates between multiple sclerosis and systemic lupus erythematosus. Clin Exp Immunol 138, 164-170.
38. Olsen, N J, Sokka, T, Seehorn, C L, Kraft, B, Maas, K, Moore, J and Aune, T M (2005) A gene expression signature for recent onset rheumatoid arthritis in peripheral blood mononuclear cells. Ann Rheum Dis 63, 1387-1392.
39. Pollack, A (2005) A special drug just for you, at the end of a long pipeline. New York Times, 08 Nov. 2005.
40. PreAnalytix Product Circular (2001) PAXgene™ blood RNA tube for the collection and stabilization of cellular RNA from whole blood.
41. Qiagen (2003) Rneasy™ MinElute™ Cleanup Handbook, for RNA cleanup and concentration with small elution volumes.
42. Qiagen Products Guide (2003) High performance RNA for gene expression analysis.
43. Schena, M, Shalon, D, Davis, R W and Brown, P O (1995) Quantitative monitoring of gene expression patterns with a complementary DNA microarray. Science 270, 467-470.
44. Sharma, P, Sahni, N S, Tibshirani, R, Skaane, P, Urdal, P, Berghagen, H, Jensen, M, Kristiansen, L, Moen, C, Sharma, P, Zaka, A, Arnes, J, Sauer, T, Akslen, L A, Schlichting, E, Boerresen-Dale, A-L, and Lönneborg, A (2005) Early detection of breast cancer based on gene-expresion patterns in peripheral blood cells.
45. Simon, R M (2004) An agenda for clinical trials: clinical trials in the genomic era. Clin Trials 1, 468-470.
46. Simon, R M (2004) When is a genomic classifier ready for prime time? Nature Clin Pract (Oncology) 1, 4-5.
47. Sinert, R. and Erogul, M. (2006) Renal Transplants. On-line reference in e-medicine (from WebMD, http://www.emedicine.com/emerg/topic607.htm)
48. Southern, E M (1975) Detection of specific sequences among DNA fragments separated by gel electrophoresis. J Mol Biol 98, 503-517.
49. Southern, E, Mir, K and Shchepinov, M. (1999) Molecular interactions on microarrays. Nature Genetics 21, 5-9.
50. U S Food and Drug Administration (2004) Stagnation or Innovation: Challenge and opportunity along the critical path to new medical products.
51. U S Food and Drug Administration (2005) Guidance for Industry: pharmacogenomic data submissions.
52. U S Food and Drug Administration (2005) Roche AmpliChip cytochrome P450 screening test and Affymterix GeneChip microarray instrumentation system—K042259 (New Device Clearance, http://www.fda.gov/cdrh/pdf4/k042259.pdf)
53. van de Vliver, M J, He, Y D, van't Veer, L J, Dai, H, Hart, A A M, Voskuil, D W, Schreiber, G J, Peterse, J L, Roberts, C, Marton, M J, Parrish, M, Atsma, D, Witteveen, A, Glas, A, Delahaye, L, van der Velde, T, Bartelink, H, Rodenhuis, S, Rutgers, E T, Friend, S H and Bernards, R (2002) A gene expression signature as a predictor of survival in breast cancer. N Engl J Med 347, 1999-2009.
54. Vernon, S D, Unger, E R, Dimulescu, I M, Rajeevan, M and Reeves, W C (2002) Utility of the blood for gene expression profiling and biomarker discovery in chronic fatigue syndrome. Disease Markers 18, 193-199.
55. Wade, Nicholas (2005) Gene variant found to triple heart risk in African-Americans. New York times, 11 Nov. 2005.
56. Wade, Nicholas (2005) Genetic find stirs debate on race-based medicine. New York Times, 11 Nov. 2005.
57. Wang, Z, Neuberg, D, Li, C, Su, L, Kim, J Y, Chen, J C, and Christiani, D C (2004) Global gene expression profiling in whole-blood samples from individuals exposed to metal fumes. Environ Health Perspect 113, 233-241.
58. Whatman FTA Protocol BD09 (2005) Removing a sample disc from an FTA® or CloneSaver™ card for analysis. pp 1-2.
59. Whatman FTA Protocol BR01 (2004) Applying and preparing blood and tissue/cell culture samples on FTA® cards for RNA analysis.
60. Whatman FTA Protocol BR01 (2005) Applying and preparing blood and tissue/cell culture samples on FTA® cards for RNA analysis. pp 1-2.
61. Whatman Product Brief (2004) Whatman FTA® for total RNA
62. Whitney, A R, Diehn, M, Popper, S J, Alizadeh, A A, Boldrick, J C, Relman, D A and Brown, P O (2003) Individuality and variation in gene expression patterns in human blood. Proc Natl Acad Sci USA 100, 1896-1901.
63. Womble, K E and Dawson, E S (2005) Isolation of mRNA and qRT-PCR from “fingerstick” whole blood on absorbant matrix. Bioventures Poster at Cold Spring Harbor Meeting, August 2005.

What we claim is:

1. A method of screening for a biological indicator in a patient comprising:
(a) collecting a biological sample from the patient;
(b) extracting genetic information from the biological sample;
(c) applying said genetic information to a genomic microarray,
wherein the microarray comprises a predefined target gene layout, wherein within said target gene layout comprises (i) multiple selected segments of multiple selected housekeeping genes to serve a baselining function and (ii) multiple selected segments of multiple selected genes specifically associated with and representing a gene profile signature for the biological indicator, wherein each segment comprises a short array of re-sequencing features;
(d) performing a gene expression assay to detect local gene sequence variations in the biological sample as compared to a prototype sequence represented on the microarray; and
(e) determining the absence or presence of the biological indicator in said patient based on the data extracted from the microarray.

2. The method of claim 1, wherein said patient is human.

3. The method of claim 1, wherein the biological sample is peripheral blood.

4. The method of claim 1, wherein the biological sample is exfoliated cells.

5. The method of claim 1, wherein said extracting comprises spotting said biological sample on a Whatman FTA Card.

6. The method of claim 1, wherein said genomic microarray is a duplicate array containing up to and including about 150 to 350 gene targets per assay slide, each in triplicate.

7. The method of claim 1, wherein said biological indicator is representative of cancer.

8. The method of claim 7, wherein said cancer is selected from the group consisting of breast cancer, colon cancer, and lung cancer.

9. The method of claim 1, wherein said biological indicator is representative of a neuromuscular or neurodegenerative disease.

10. The method of claim 9, wherein said neuromuscular or neurodegenerative disease is selected from the group consisting of multiple sclerosis, Alzheimer's disease, and muscular dystrophy.

11. The method of claim 1, wherein said biological indicator is representative of a response to an occupational or incidental exposure.

12. The method of claim 11, wherein said occupational or incidental exposure is selected from the group consisting of radiation, metal fumes, benzene, and tobacco.

13. The method of claim 1, wherein said biological indicator is representative of a cardiovascular disease or stroke.

14. The method of claim 13, wherein said cardiovascular disease or stroke is selected from the group consisting of stroke, heart failure, and hypertension.

15. The method of claim 1, wherein said biological indicator is representative of a response following organ transplantation.

16. The method of claim 15, wherein said organ transplantation is selected from the group consisting of liver transplant and kidney transplant.

17. The method of claim 1, wherein said genetic information is DNA.

18. The method of claim 1, wherein said genetic information is mRNA.

19. A method of differential diagnosis and detection of a biological indicator in a patient comprising:
(a) collecting a biological sample from the patient;
(b) extracting genetic information from the biological sample;
(c) recovering genetic information from a biological sample obtained from said patient at a time predating said collecting and which was stored so as to preserve the structural integrity of said genetic information;
(c) applying said genetic information obtained in (b) to one assay slide of a genomic microarray which is a duplicate array containing up to and including about 150 to 350 gene targets per assay slide, each in triplicate, and applying said genetic information obtained in (c) to the other assay slide of said genomic microarray,
wherein the microarray comprises a predefined target gene layout, wherein within said target gene layout comprises (i) multiple selected segments of multiple selected housekeeping genes to serve a baselining function and (ii) multiple selected segments of multiple selected genes specifically associated with and representing a gene profile signature for the biological indicator, wherein each segment comprises a short array of re-sequencing features; and
(d) performing a gene expression assay to detect local gene sequence variations in the biological sample by comparing the gene expression profile for the genetic information obtained in (b) to the genetic information obtained in (c).

20. The method of claim 19, further comprising:
(e) correlating the difference between said comparing to the absence or presence of a disorder represented by said biological indicator.

21. The method of claim 19, further comprising:
(i) repeating (a) and (b) after a predetermined time interval
(ii) commencing (c)-(e); and
(iii) determining whether the etiology underlying said biological indicator is progressing or improving on the basis of said comparing and cross-correlation to prior implementation of (c)-(e).

22. The method of claim 19, wherein said patient is human.

23. The method of claim 19, wherein the biological sample is peripheral blood.

24. The method of claim 19, wherein the biological sample is exfoliated cells.

25. The method of claim 19, wherein said extracting comprises spotting said biological sample on a Whatman FTA Card.

26. The method of claim 19, wherein said biological indicator is representative of cancer.

27. The method of claim 26, wherein said cancer is selected from the group consisting of breast cancer, colon cancer, and lung cancer.

28. The method of claim 19, wherein said biological indicator is representative of a neuromuscular or neurodegenerative disease.

29. The method of claim 28, wherein said neuromuscular or neurodegenerative disease is selected from the group consisting of multiple sclerosis, Alzheimer's disease, and muscular dystrophy.

30. The method of claim 19, wherein said biological indicator is representative of a response to an occupational or incidental exposure.

31. The method of claim 30, wherein said occupational or incidental exposure is selected from the group consisting of radiation, metal fumes, benzene, and tobacco.

32. The method of claim 19, wherein said biological indicator is representative of a cardiovascular disease or stroke.

33. The method of claim 32, wherein said cardiovascular disease or stroke is selected from the group consisting of stroke, heart failure, and hypertension.

34. The method of claim 19, wherein said biological indicator is representative of a response following organ transplantation.

35. The method of claim 34, wherein said organ transplantation is selected from the group consisting of liver transplant and kidney transplant.

36. The method of claim 19, wherein said genetic information is DNA.

37. The method of claim 19, wherein said genetic information is mRNA.
